(19) United States
(12) Patent Application Publication
(10) Pub. No.: US 2006/0195191 A1
(43) Pub. Date: Aug. 31, 2006

(54) Modular disc device

(75) Inventors: Thomas M. Sweeney II, Sarasota, FL (US); David T. Hawkes, Pleasant Grove, UT (US); Michael D. Ensign, Salt Lake City, UT (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Alphaspine Inc., Sarasota, FL (US)

(21) Appl. No.: 11/328,498

(22) Filed: Jan. 9, 2006

Related U.S. Application Data
(60) Provisional application No. 60/642,256, filed on Jan. 8, 2005.
(60) Provisional application No. 60/687,500, filed on Jun. 3, 2005.
(60) Provisional application No. 60/687,185, filed on Jun. 3, 2005.
(60) Provisional application No. 60/687,498, filed on Jun. 3, 2005.
(60) Provisional application No. 60/687,499, filed on Jun. 3, 2005.

(51) Int. Cl. A61F 2/44

(52) U.S. Cl. 623/17.13; 623/17.11

(57) ABSTRACT

The disclosure generally relates to a modular disc that is insertable by sections into a disc space located between vertebral bodies. The primary segments of the modular disc are one or more middle members engageable with first and second side members. The first and second side members are successively inserted into and positioned in the disc space one at a time. The middle member is then slidably engaged on rails extending from the respective first and second side members. Eventually, the middle member is moved along the guides and positioned between the respective first and second side members and the guides or rails are detached. Optionally, a cushioning and/or biasing member may be located between upper and lower portions of the side members and/or middle member.

### CROSS-REFERENCES TO RELATED APPLICATIONS

This application claims the benefit under 35 U.S.C. §119(e) of U.S. Provisional Patent Application Nos. 60/642,256 filed Jan. 8, 2005, 60/622,097 filed Oct. 25, 2004, 60/687,500 filed Jun. 3, 2005, 60/687,185 filed Jun. 3, 2005, 60/687,498 filed Jun. 3, 2005, and 60/687,499 filed Jun. 3, 2005, where these provisional applications are also incorporated herein by reference in their entireties.

### BACKGROUND OF THE INVENTION

1. Field of the Invention

The present invention relates generally to a modular disc that is insertable between vertebral bodies and functions as an artificial replacement intervertebral disc for a spine.

2. Description of the Related Art

The intervertebral discs are the cushions that act as shock absorbers between each of the vertebra in a person's spine. There is one disc between each pair of the vertebrae. Each disc has a strong outer ring of fibers called the annulus and a soft, jelly-like center called the nucleus pulposus. The annulus is the outermost and strongest portion of the disc. The annulus is also a ligament and functions to connect the vertebrae together. The nucleus of the disc serves as the main shock absorber.

Intervertebral discs become more rigid with age, which means that the elasticity of the disc decreases and makes it more vulnerable to injury. One type of injury that may occur to an intervertebral disc is a herniated disc or rupture of the intervertebral disc. A herniated disc occurs when the annulus becomes damaged and allows the soft inner material of the nucleus pulposus to rupture out of a disc space defined by the annulus. If the annulus tears near the spinal canal, the nucleus pulposus material can push into the spinal canal. When a herniated disc bulges out from between the vertebral bodies and from the annulus, the spinal nerves and spinal cord can become pinched and/or compressed.

Artificial disc replacement is designed to restore the normal disc height by replacing at least a portion of a damaged intervertebral disc with an artificial disc implant. There are several types of artificial discs available including those fabricated from combinations of metal, polyethylene, polyurethane, and other biomaterials. Some of these artificial discs are referred to by the trade names of FlexiCore®, ProDisc®, and Maverick™.

Although artificial intervertebral discs have been used internationally for over a decade, only the Charité™ artificial disc has received the approval of the Food and Drug Administration (FDA). The Charité™ artificial disc is a three-piece articulating medical device with a sliding core sandwiched between two metal endplates. The sliding core is made from plastic and the endplates are made from a cobalt chromium alloy. The endplates support the core and have small teeth or barbs that secure them to the vertebrae above and below the disc space. The sliding core fits between the endplates.

### BRIEF SUMMARY OF THE INVENTION

The embodiments described herein are generally related to a modular disc that can be used as a replacement for a natural intervertebral disc. The modular disc is insertable into a disc space of a patient in segments. The modular disc comprises a middle member disposed between and engageable with respective side members. The side members are inserted into and positioned in the disc space one at a time. The middle member is slidably engaged and disposed between the respective side members. Optionally, a cushioning and/or biasing member may be located between upper and lower portions of the side members and/or middle member.

In one aspect, a modular disc includes a middle member having engagement portions; and first and second side members having complementary engagement portions, the middle member disposed between the first and second side members, wherein the middle member is slidably engageable with the first and second side members.

In another aspect, a modular disc includes a middle member having an engagement portion; and a first side member having a first complementary engagement portion comprising a first portion and a second portion, the first portion positioned proximate the first side member, the second portion extending radially from an outer circumference of the first side member, wherein the engagement portion of the middle member is slidably engageable with the first and second portions of the complementary engagement portion of the first side member.

In yet another aspect, a method of inserting a spinal disc between vertebral bodies includes inserting a first side member into a first portion of a disc space located between the vertebral bodies, the first side member having an engagement member with a first engagement portion and a second engagement portion, the first engagement portion coupled to the first side member and locatable within the disc space, the second engagement portion extending from the first engagement portion and protrudable from the disc space in a first direction; inserting a second side member into a second portion of the disc space, the second side member having an engagement member with a first engagement portion and a second engagement portion, the first engagement portion coupled to the second side member and locatable within the disc space, the second engagement portion extending from the first engagement portion and protrudable from the disc space in substantially the first direction; slidably engaging middle members with the second engagement portions of the first and second side members; sliding the middle members along the second engagement portions of the first and second side members; sliding the middle members along the first engagement portions of the first and second side members until the middle members are located within the disc space; and detaching the second engagement portions of the first and second side members.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility.

FIG. 1 is an isometric view of a modular disc having a middle member disposed between side members, according to one illustrated embodiment.

FIG. 2 is a top plan view of the modular disc of FIG. 1 after extendable engagement portions of the side members have been removed.

FIG. 3 is a cross-sectional view of the modular disc of FIG. 1 taken along line 3-3 of FIG. 2.

FIG. 4 is a detailed view of an engagement between one of the middle members and one of the side members of the modular disc of FIG. 1.

FIG. 5 is a top plan view of the modular disc of FIG. 1 before the extendable engagement portions of the side members have been removed.

FIG. 6 is a cross-sectional view of another modular disc illustrating an alternative engagement of the middle members to the side members.

FIG. 7 is an isometric view of a modular disc having a biasing member disposed between side members, according to one illustrated embodiment.

FIG. 8 is a front side elevational view of the modular disc of FIG. 7.

FIG. 9 is a right side elevational view of the modular disc of FIG. 7.

FIG. 10 is a bottom, left isometric view of the modular disc of FIG. 7 before extendable engagement portions of the side members have been removed.

FIG. 11 is a side elevational view of a portion of a spine showing a number of vertebral bodies separated by intervertebral discs.

FIG. 12 is a schematic view of a surgical site showing an annulus of an intervertebral disc with a disc space and an access region.

FIG. 13 is a schematic view of first side members of a modular disc being inserted in through the access region and into the disc space of the annulus.

FIG. 14 is a schematic view of first side members and second side members positioned in the disc space of the annulus.

FIG. 15 is a schematic view of middle members of a modular disc being slidably engaged with rail guides extending from the first and second side members of the modular disc.

FIG. 16 is a schematic view of an assembled modular disc positioned within the disc space of the annulus and having rail guides extending out of the disc space through the access region of the annulus.

FIG. 17 is a schematic view of an assembled modular disc positioned within the disc space of the annulus and with the rail guides of FIG. 16 detached from the modular disc.

### DETAILED DESCRIPTION OF THE INVENTION

In one embodiment, a modular disc can be used as a replacement for a natural intervertebral disc. The modular disc is insertable into a disc space of a patient in segments or sections. The primary segment of the modular disc is a middle member, which may comprise one or two pieces, and which is engageable with first and second side members. The first and second side members are successively inserted into and positioned in the disc space one at a time. The middle member is then slidably engaged with guides or rails extending from the respective first and second side members. The middle member is moved along the guides and positioned between the respective first and second side members and the extending guides or rails are removed. Optionally, a cushioning and/or biasing member may be located between upper and lower portions of the side members and/or between the first and second pieces of the middle member.

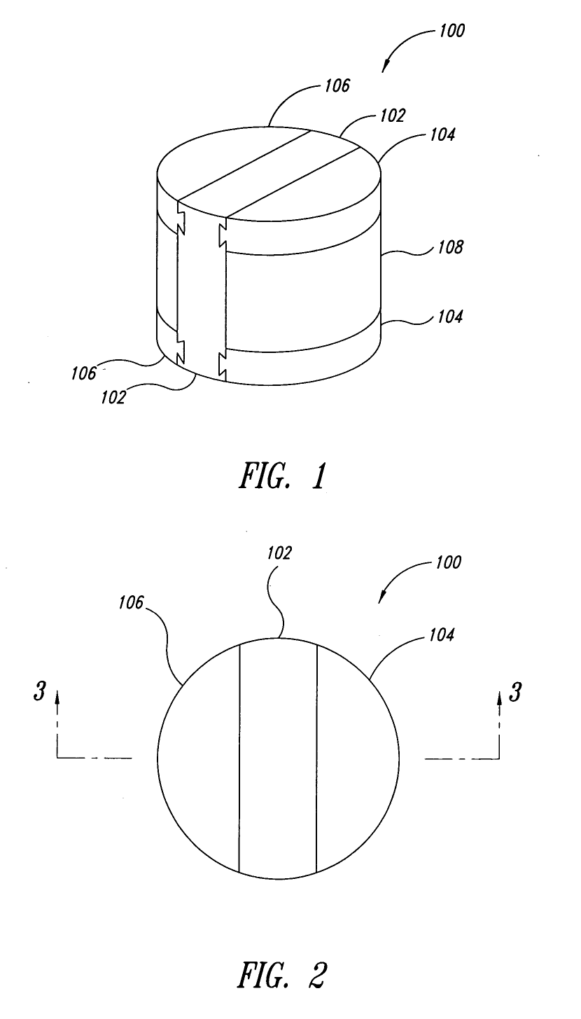

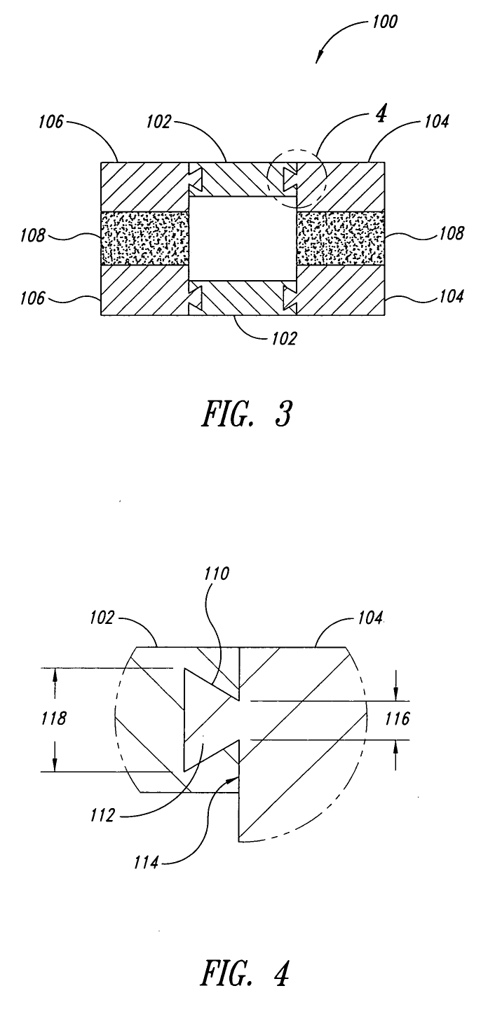

FIGS. 1 and 2 show a modular disc 100 having middle member 102, first side members 104, and second side members 106, according to one illustrated embodiment. In addition, the modular disc 100 may optionally include a cushioning, damping, biasing, and/or otherwise resilient member 108 disposed between upper and lower portions of the side members 104, 106. In one embodiment the cushioning member 108 is an elastomeric material. In another embodiment, the cushioning member 108 is a biasing member, which will be described in greater detail below. The upper and lower surfaces of the modular disc 100 may be hardened or coated to achieve an improved bearing surface with the vertebral bodies.

FIGS. 3 and 4 show the modular disc 100 with a two-piece middle member 102 engageably keyed to the first side members 104 and the second side members 106. In FIG. 4, engagement portions 110 of the middle member 102 comprise a recessed or keyed slot 110. The engagement portions 110 of the middle member are sized to closely, yet slidably, receive complementary engagement portions 112 of the first side members 104.

FIG. 4 shows a detailed view of one engagement portion 110 of the middle member 102 coupled to one complementary engagement portion 112 of one first side member 104. In the illustrated embodiment, the complementary engagement portion 110 of the first side member is a protuberance or key 112 located proximate to and extending adjacently from a first surface 114 of the first side member 104. The protuberance 112 includes a narrow base 116 proximate the first surface 114. The protuberance 112 extends or flares out to a wide portion 118 as it extends from the first surface 114 to form an interlocking key 112 that is engageable with the engagement portion 110 of the middle member 102. In one embodiment, the protuberance 112 and the engagement portion 110 operate as dovetail portions for adjoining the components together. In an alternate embodiment, the side members 104, 106 may include slots and the middle member 102 may include protuberances.

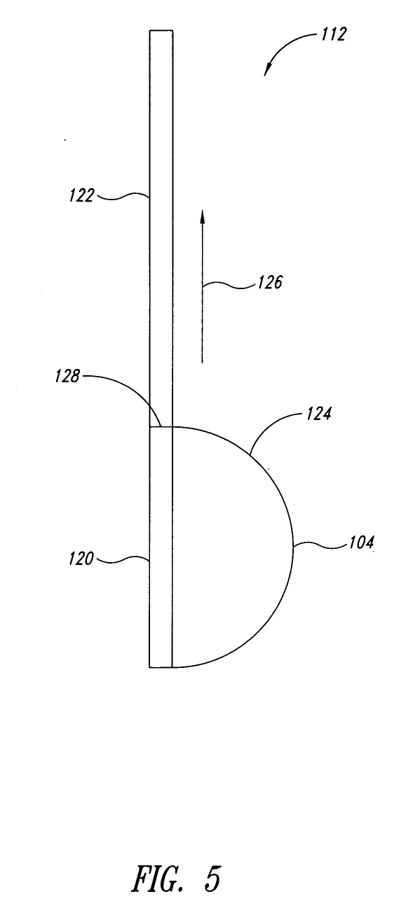

FIG. 5 shows the first side member 104, which is representative of the other side members, where the complementary engagement portion 112 includes a first portion 120 and a second portion 122. The first portion 120 is positioned adjacent and proximate to the first side member 104. The second portion 122 extends radially from an outer circumference 124 of the first side member 104 in a first direction 126. In addition, the complementary engagement portion 112 may include a reduced shear strength region 128 to allow the second portion 122 to be easily separated from the first portion 120 after the first side member 104 has been placed in a disc space of a patient. The installation of the modular disc 100 and the purpose of having both the first portion 120 and the second portion 122 of the complementary engagement portion 112 is described in greater detail below.

FIG. 6 shows a modular disc 200 having a two-piece middle member 202, first side members 204, second side members 206, and a cushioning member 208. The middle member 202 includes engagement portions 210. The side members 204, 206 include complementary engagement portions 212, which are formed as bulbous protuberances 212 extending from a first surface 214.

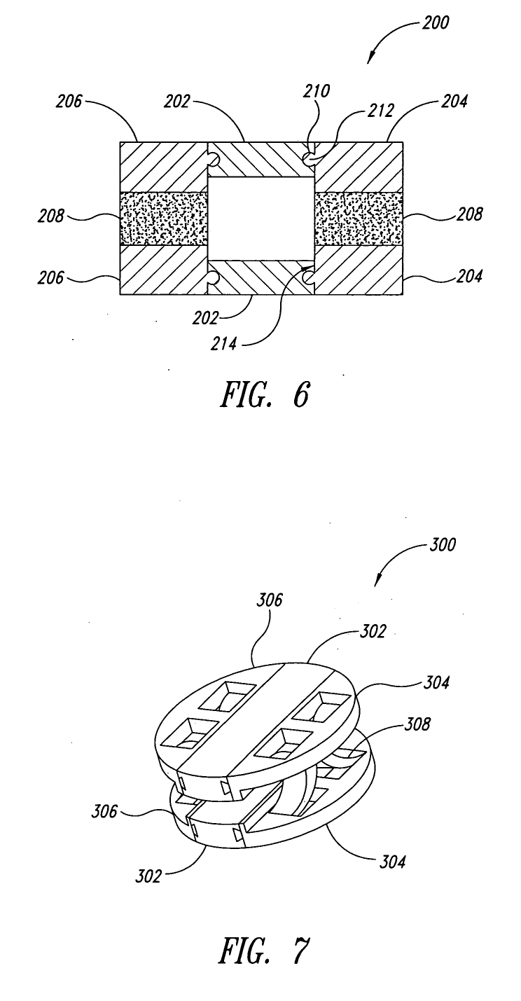

FIG. 7 shows another modular disc 300 comprising a two-piece middle member 302, first side members 304, second side members 306, and biasing members 308. The biasing members 308 are disposed between and coupled to the upper and lower portions of the respective first and second side members 304, 306. The biasing members 308 can be springs, damping members, or some combination of a spring/damper device. In one embodiment, the biasing members 308 comprise leaf springs, Belleville springs, or coil springs. The biasing members 308 are capable of compressing, but may also extend, when reacting to static and dynamic loading of the spine. This loading may cause the vertebral bodies to be forced closer together (i.e., biasing is urged into compression) or pulled further apart (i.e., biasing is urged into extension) from one another. The biasing members 308 react to such loading and then return the vertebral bodies back to a neutral or a rest position when the loading is complete. In addition, the biasing members 308 couple the upper and lower portions of the side members together, which increases stability of the modular disc 300 while under load.

Alternatively, the biasing members 308 may comprise at least one spacer insertable between the upper and lower side members 304, 306 and/or between the upper and lower middle members 302. The at least one spacer may be an articulating spacer that complementarily contacts at least some portion of the inner surfaces of the middle and/or side members. Accordingly, the articulating spacer would permit the upper and lower portions of the modular disc 300 to rotate independently of one another.

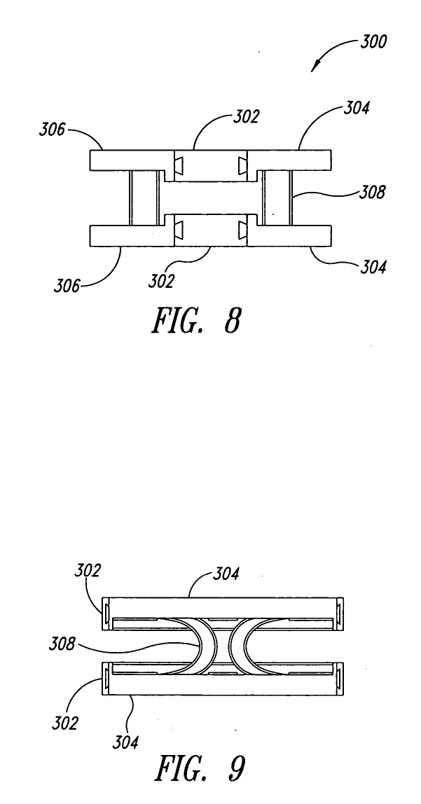

FIGS. 8 and 9 show respective side views of the modular disc 300. FIG. 8 shows that the middle members 302 are engaged with the side members 304, 306 in a manner similar to that described in the previous embodiment. FIG. 9 shows the biasing members 308 comprising a curved leaf spring coupled to upper and lower portions of the respective first and second side members 304, 306. Additionally or alternatively, a biasing member (not shown) may be disposed between and coupled to the upper and lower middle members 302.

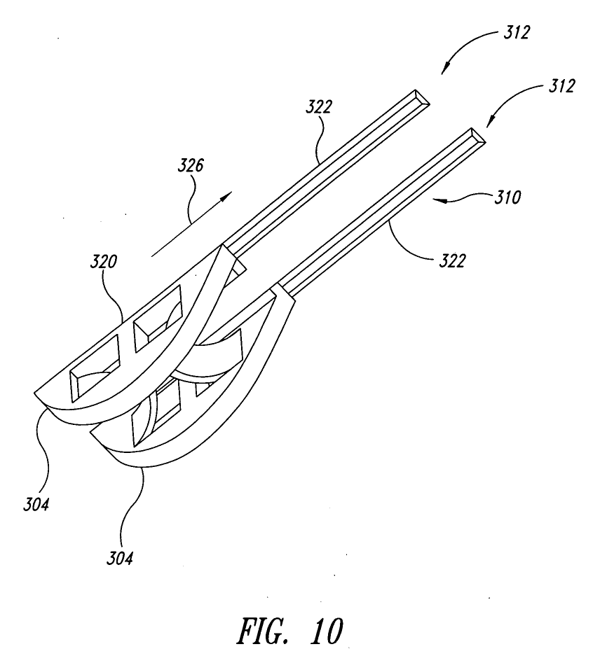

FIG. 10 shows the first side members 304 and a biasing member 308 of the modular disc 300 coupled together. The first side members 304 each include a complementary engagement portion 312, which is in a pre-installed state. The complementary engagement portions 312 include a first portion 320 and a second portion 322. The first portion 320 is positioned adjacent and proximate to the respective first side member 304. The second portion 322 extends radially from an outer circumference 324 of the respective first side member 304 in a first direction 326.

Installation of a Modular Disc

FIG. 11 shows a portion of a spine 400 having vertebral bodies 402 and intervertebral discs 404, which act as cushions between the bodies 402. Each disc includes a tough, fibrous outer layer called an annulus and a softer center portion called a nucleus. During a surgical procedure to insert a modular disc, the nucleus is removed, but the annulus is kept in place.

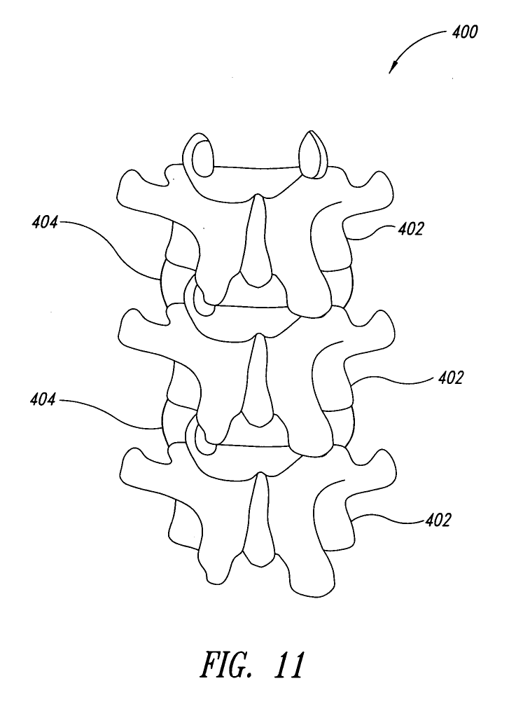

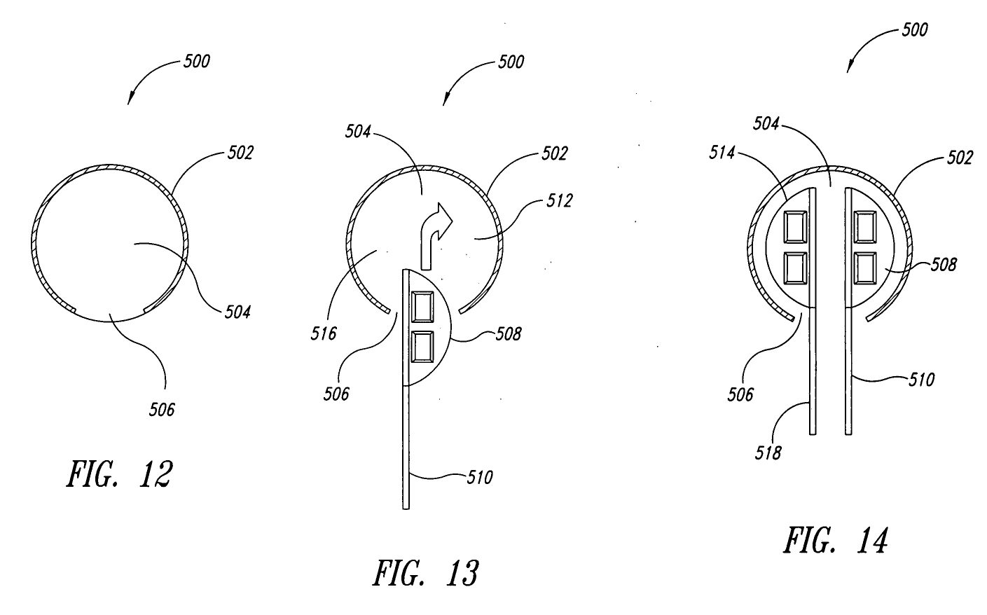

FIG. 12 shows a spinal surgical site 500 where an annulus 502 has been prepared to receive the modular disc. The annulus 502 includes a disc space 504 and an access region 506 to receive the modular disc.

FIG. 13 shows first side members 508 having radially extending guides 510 being inserted through the access region 506. The first side members 508 are positioned in a first region 512 of the disc space 504. Second side members 514 (FIG. 14) are inserted and positioned in a second region 516 of the disc space 504.

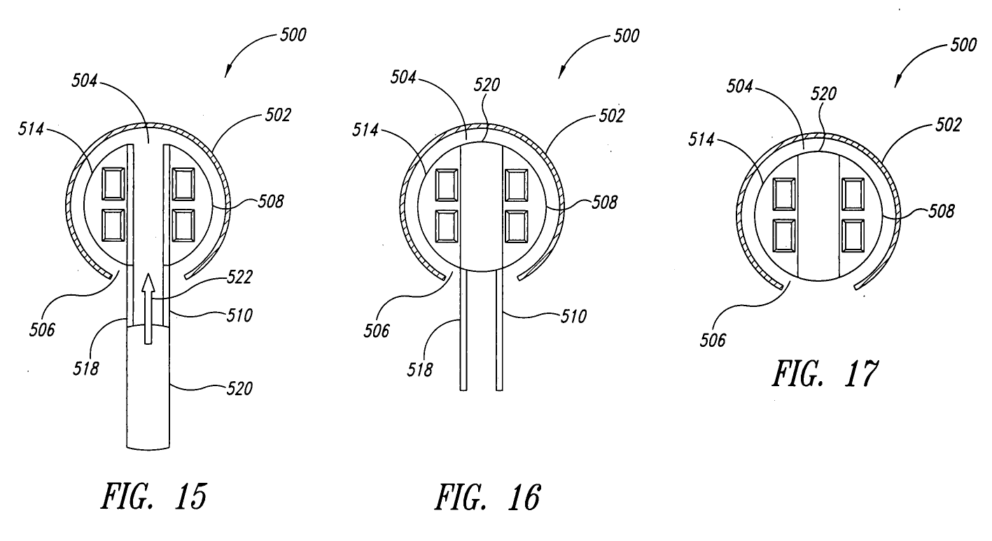

FIG. 14 shows the first side members 508 and the second side members 514 positioned in the disc space 502. The radially extending guides 510 of the first side members 508 and radially extending guides 518 of the second side members 514 protrude from the access region 506 of the annulus 502 and may be adjusted to receive middle member 520 (FIG. 15).

FIG. 15 shows the middle members 520 slidably engaging the radially extending guides 510, 518 of the respective side members 508, 514. The middle member 520 is pushed onto the guides 510, 518 toward the access region 506 of the annulus 502 as indicated by the arrow 522.

FIG. 16 shows the middle member 520 coupled with the side members 508, 514 after the middle member 520 has been slid down the guides 510, 518 and into the disc space 504 of the annulus 502. The guides 510, 518 remain attached and protruding from the side members 508, 514 and protruding out through the access region 506 of the annulus 502.

FIG. 17 shows the assembled and installed modular disc 524 after the guides 510, 518 have been detached from the respective side members 508, 514. In one embodiment, the guides 510, 518 are snapped, broken, or clipped off near the perimeter of the modular disc 524.

The modular discs described herein are installed in sections, for example the first side member, the second member, and then the middle member. This sectional installation approach may advantageously permit an opening to the surgical site to be smaller because the surgeon does not need to separate and/or remove a lot of tissue to maneuver the modular disc section through the body to the surgical site. This may reduce the amount of trauma to the tissue near the surgical site, which in turn may allow a faster recovery time for the patient. One other aspect of having a smaller surgical opening is that the modular disc may be inserted using a posterior (i.e., through a patient's back) or lateral (i.e., through a patient's side) approach. This other aspect provides an advantage over other spinal replacement discs because those discs are sized to be installed using an anterior approach, which results in a larger surgical opening through a patient's stomach. It is generally understood the access to the spine via the posterior or lateral approach is more limited than via the anterior approach.

In addition, the components of the modular disc are coupled together during assembly/installation of the disc, thus these components operate to provide a stable structure with redundant and/or backup load paths (e.g., multiple biasing members).

The above description of illustrated embodiments, including what is described in the Abstract, is not intended to be exhaustive or to limit the invention to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to various disc devices, not necessarily the exemplary disc devices generally described above.

The various embodiments described above can be combined to provide further embodiments. All of the U.S. patents, U.S. patent application publications, U.S. patent applications, foreign patents, foreign patent applications, and non-patent publications referred to in this specification and/or listed in the Application Data Sheet, to include U.S. patent application Ser. No. 11/260,880 filed Oct. 25, 2005; U.S. Provisional Patent Application Nos. 60/642,256 filed Jan. 8, 2005; 60/622,097 filed Oct. 25, 2004;, 60/687,500 filed Jun. 3, 2005;, 60/687,185 filed Jun. 3, 2005;, 60/687,498 filed Jun. 3, 2005;, and 60/687,499 filed Jun. 3, 2005; are incorporated herein by reference, in their entireties. Aspects of the invention can be modified, if necessary, to employ aspects of other disc devices and concepts of the various patents, applications, and publications to provide yet further embodiments of the invention.

These and other changes can be made to the invention in light of the above-detailed description. In general, in the following claims, the terms used should not be construed to limit the invention to the specific embodiments disclosed in the specification and the claims, but should be construed to include all spinal disc devices that operate in accordance with the claims. Accordingly, the invention is not limited by the disclosure, but instead its scope is to be determined entirely by the following claims.

1. A modular disc comprising:
a middle member having engagement portions; and
first and second side members having complementary engagement portions, the middle member disposed between the first and second side members, wherein the middle member is slidably engageable with the first and second side members.

2. The modular disc of claim 1 wherein the engagement portions of the middle member comprise recessed slots.

3. The modular disc of claim 1 wherein the engagement portions of the middle member comprise keyed slots.

4. The modular disc of claim 1 wherein the engagement portions of the middle member comprise dovetail portions extending laterally therefrom.

5. The modular disc of claim 1 wherein the complementary engagement portions of the first and second side members comprise recessed slots.

6. The modular disc of claim 1 wherein the complementary engagement portions of the first and second side members comprise keyed slots.

7. The modular disc of claim 1 wherein the complementary engagement portions of the first and second side members comprise dovetail portions extending laterally therefrom.

8. The modular disc of claim 1 wherein the complementary engagement portions of the first and second side members comprise guide tracks extending therefrom.

9. The modular disc of claim 1 wherein the first and second side members include upper and lower portions.

10. The modular disc of claim 1, further comprising:
an intermediate member disposed between the upper and lower portions of the first and second side members.

11. The modular disc of claim 10 wherein the intermediate member is a biasing member.

12. The modular disc of claim 11 wherein the biasing member is a spring.

13. The modular disc of claim 10 wherein the intermediate member is a damping member.

14. The modular disc of claim 13 wherein the damping member is an elastomeric member.

15. The modular disc of claim 10 wherein the intermediate member is a spacer.

16. The modular disc of claim 1, further comprising:
an intermediate member disposed between the upper and lower portions of the middle member.

17. The modular disc of claim 1 wherein the middle member is a two-piece middle member.

18. A modular disc comprising:
a middle member having an engagement portion; and
a first side member having a first complementary engagement portion comprising a first portion and a second portion, the first portion positioned proximate the first side member, the second portion extending radially from an outer circumference of the first side member, wherein the engagement portion of the middle member is slidably engageable with the first and second portions of the complementary engagement portion of the first side member.

19. The modular disc of claim 18 wherein the engagement portion of the middle member comprises a recessed slot.

20. The disc of claim 18 wherein the engagement portion of the middle member comprises a keyed slot.

21. The modular disc of claim 18 wherein the engagement portion of the middle member comprises a dovetail portion.

22. The modular disc of claim 18 wherein the complementary engagement portion of the first side member comprises a recessed slot.

23. The modular disc of claim 18 wherein the complementary engagement portion of the first side member comprises a keyed slot.

24. The modular disc of claim 18 wherein the complementary engagement portion of the first side members comprises a dovetail extension.

25. The modular disc of claim 18 wherein the complementary engagement portion of the first side member comprise a guide track.

26. The modular disc of claim 18, further comprising:
an intermediate member positioned adjacent to the first side member.

27. The modular disc of claim 26 wherein the intermediate member is a biasing member.

28. The modular disc of claim 27 wherein the biasing member is a spring.

29. The modular disc of claim 26 wherein the intermediate member is a damping member.

30. The modular disc of claim 29 wherein the damping member is an elastomeric member.

31. The modular disc of claim 26 wherein the intermediate member is a spacer.

32. The modular disc of claim 18 wherein the complementary engagement portion includes a decreased strength region located proximate the outer circumference of the first side member.

33. A method of inserting a spinal disc between vertebral bodies, the method comprising:
inserting a first side member into a first portion of a disc space located between the vertebral bodies, the first side member having an engagement member with a first engagement portion and a second engagement portion, the first engagement portion coupled to the first side member and locatable within the disc space, the second engagement portion extending from the first engagement portion and protrudable from the disc space in a first direction;
inserting a second side member into a second portion of the disc space, the second side member having an engagement member with a first engagement portion and a second engagement portion, the first engagement portion coupled to the second side member and locatable within the disc space, the second engagement portion extending from the first engagement portion and protrudable from the disc space in substantially the first direction;
slidably engaging a middle member with the second engagement portions of the first and second side members;
sliding the middle member along the second engagement portions of the first and second side members;
sliding the middle member along the first engagement portions of the first and second side members until the middle member is located within the disc space; and
detaching the second engagement portions of the first and second side members.

34. The method of claim 33 wherein detaching the second engagement portions of the first and second side members includes breaking off the second engagement portions along a reduced strength region proximate the first engagement portions.

35. The method of claim 33 wherein inserting the spinal disc between vertebral bodies includes inserting the spinal disc through a posterior surgical opening.

36. The method of claim 33 wherein inserting the spinal disc between vertebral bodies includes inserting the spinal disc through a lateral surgical opening.
